(19) United States
(12) Patent Application Publication
(10) Pub. No.: US 2022/0277858 A1
(43) Pub. Date: Sep. 1, 2022

(54) Medical Prediction Method and System Based on Semantic Graph Network

(71) Applicant: Beijing University of Technology, Beijing (CN)

(72) Inventors: Qing Zhao, Beijing (CN); Jianqiang Li, Beijing (CN); Dezhong Xu, Beijing (CN); Chun Xu, Beijing (CN)

(21) Appl. No.: 17/329,657

(22) Filed: May 25, 2021

(30) Foreign Application Priority Data
Feb. 26, 2021 (CN) 2021102190693

(51) Int. Cl. G16H 50/70; G06F 40/169; G16H 50/20; G06F 40/295; G06F 40/30; G06N 5/02; G06K 9/62

(52) U.S. Cl. CPC G16H 50/70; G06F 40/169; G16H 50/20; G06F 40/295; G06F 40/30; G06N 5/02; G06K 9/629; G06K 9/6292

(57) ABSTRACT

The present invention discloses a medical prediction method and system based on a semantic graph network, which recognizes an entity in an electronic medical record based on domain knowledge, and uses a two-way gated loop unit to learn a sequence features of a text. Secondly, in order to extract a semantic relation in the electronic medical record in a fine-granularity manner, the present invention defines two types of subgraphs, graph representation based on defined knowledge and graph representation based on undefined knowledge, and uses a Graph Convolution Network (GCN) and a Graph Attention Network (GAT) to extract a semantic relation representation, where the graph representation based on undefined knowledge allows the learning of a relation between an entity or an word and the graph representation based on undefined knowledge, and it also allows to learn a relation between word or entity and itself, in order to translate entity or word representation into a uniform graph embedding representation. For an attribute-value pair, the present invention uses a bi-directional gate recurrent unit (Bi-GRU) to extract an entity corresponding to a numerical feature or a categorical feature after extracting the numerical feature or the categorical feature in the electronic medical record to construct attribute-value graph representation. Finally, the semantic relation and an attribute-value are fused to train a prediction model of a disease level.

### CROSS REFERENCE TO RELATED APPLICATION

The present invention belongs to the field of computer technology, and particularly relates to a medical prediction method and system based on a semantic graph network.

### BACKGROUND OF THE INVENTION

Chronic diseases are the main type of diseases that threaten human life. However, since most chronic diseases are preventable and treatable, early intervention can effectively reduce the aggravating probability of the chronic diseases. Establishing a prediction model to analyze the status of a patient to predict the future development of the disease of the patient is an important prerequisite for preventive care and reducing the burden of the chronic disease on an individual.

With the widespread use of an electronic medical record, a disease prediction model based on semantic analysis has made certain development. Currently, a method of constructing a prediction model based on an electronic medical record is mainly divided into two categories: (1) a hypothesis-driven method. The principle of the hypothesis-driven method is to start with the hypothesis proposed by a clinical expert based on observations and clinical experience, and then find out facts from medical data. Deductive reasoning is used to verify the authenticity of the hypothesis. And the prediction model is derived from a set of validated hypotheses. Generally speaking, the hypothesis-driven method cannot make full use of valuable information contained in medical data. (2) A data-driven method. The principle of the data-driven method is to use a fully labeled medical data set to train a machine learning model to achieve disease prediction. However, traditional machine learning models require domain experts to specify clinical features in a special way, and the success of the final prediction model largely depends on the complex supervision of hand-designed feature selection. For example, Effective Heart Disease Prediction Using Hybrid Machine Learning Techniques published by Senthilkmar Mohan et al. in 2019 proposed a linear mixed random forest model for predicting a heart disease. Deep learning can reduce the complexity of traditional machine learning feature selection, automatically learn deeper features from data and has become the main method of the prediction model.

A method for predicting a disease based on the deep learning usually uses words or concept vectors as the main feature representation of medical texts. For example, the Augmenting Embedding with Domain Knowledge for Oral Disease Diagnosis Prediction published by Guangkai Li, Songmao Zhang, et al. in SmartCom 2018 learned the concepts of symptoms related to diagnoses from the domain ontology and used neural networks to learn conceptual features in electronic medical records to construct a prediction model of an oral disease. However, in the electronic medical record, many entities or words express disease-related information through semantic relation. For example, “a patient suffered from chest oppression and wheezing after exercise 3 years ago, and were diagnosed as chronic obstructive pulmonary disease (COPD) in our hospital.” If an attribute-value “COPD—3 years ago” was not considered, it was difficult to distinguish whether COPD is a past disease or a current disease. Another example is “a patients uses Seretide to improve wheezing symptom.” If a doctor only consider an entity representation without considering an entity relation, the true meaning expressed in the sentence cannot be discovered. In addition, most clinical medical decisions are made based on a test-test result.

Therefore, finding a medical prediction method and system based on a semantic graph network has become researchers' concern.

### SUMMARY OF THE INVENTION

In order to solve the forgoing technical problems, the present invention provides a medical prediction method and system based on a semantic graph network for disease classification. An entity in an electronic medical record is recognized based on a domain, and a two-way gated loop unit is used to learn a sequence feature of a text. Secondly, in order to extract semantic relation in the electronic medical record in a fine-granularity manner, the present invention defines two types of subgraphs, graph representation based on defined knowledge and graph representation based on undefined knowledge, and uses a Graph Convolution Network (GCN) and a Graph Attention Network (GAT) to extract a semantic relation representation, where the graph representation based on undefined knowledge allows the learning of a relation between words or an entity and a word and graph representation based on undefined knowledge it also allows to learn a relation between word or entity and itself, in order to translate entity or word representation into a uniform graph embedding representation. For an attribute-value pair the present invention uses a bi-directional gate recurrent unit (Bi-GRU) to extract an entity corresponding to a numerical feature or a categorical feature after extracting the numerical feature or the categorical feature in the electronic medical record to construct attribute-value graph representation. Finally, the semantic relation and an attribute-value are fused to train a prediction model of a disease level.

In order to solve the forgoing technical problems, the present invention proposes a medical prediction method based on a semantic graph network, specifically including the following steps:

S1. Preprocessing medical text data.
S2. Feature extraction on the preprocessed medical text data.
S3. Fusing a multi-granularity feature on the extracted feature to obtain a final document feature representation.
S4. Predicting a chronic disease on the final document feature representation.

Preferable, Step S1 is specifically as follows:

S11. Manually annotating the medical text data according to a target category that needs to be predicted, and loading the medical text data into a domain ontology.
S12. Cutting the medical text data into Chinese character strings according to punctuation marks, numbers and space characters, and removing off-stream words.
Preferably, the feature extraction in Step S2 includes: entity embedding representation, word embedding representation, semantic relation representation extraction, and attribute-value pair extraction.
Preferably, the entity embedding representation is specifically as follows:
First, mapping the preprocessed medical text data to the domain ontology; dividing the medical text data into a semantic set via a maximum matching method; then finding an entity set matching the semantic set and an entity type set corresponding to the entity set from the semantic set to obtain an entity representation and an entity type representation; and finally, combining the entity representation and the entity type representation to extract an entity representation.
Preferably, the word feature embedding representation and the attribute-value pair extraction are specifically as follows:

Using a Bi-GRU to find a dependency relation between word sequences in the medical text data, and putting sequence information between words into a graph attention network to identify semantic relation and extract an attribute-value pair. Preferably, the semantic relation representation extraction is specifically as follows:

using a graph convolution network and the graph attention network to construct a semantic relation graph and defining two types of subgraphs of graph representation based on defined knowledge and graph representation based on undefined knowledge, where the graph representation based on defined knowledge uses a relation between entities marked in the domain ontology and uses the graph convolution network and the graph attention network to extract an entity relation in an electronic medical record text. For the entity or the word whose corresponding relation cannot be found from the domain ontology, the graph representation based on undefined knowledge directly uses the graph convolution network and the graph attention network to extract a relation between the words or the entities based on a dependency relation between words in context extracted by the Bi-GRU.

Preferable, Step S3 is specifically as follows:

Feature fusing entity feature embedding representation, word embedding representation feature, an semantic relation representation, and attribute-value pair representation to obtain the final document feature representation.

Preferable, Step S4 is specifically as follows:

Inputting the document feature representation into softmax layer for medical prediction, and calculating a loss function based on a cross entropy between a real label and a predicted label to obtain a classification result of a disease type and a prediction result of a disease level.

A medical prediction system based on a semantic graph network includes a data preprocessing module, a feature extraction module, a multi-granularity feature fusion module, and a disease type classifier module.

An output terminal of the data preprocessing module is connected to an input terminal of the feature extraction module. An output terminal of the feature extraction module is connected to an input terminal of the multi-granularity feature fusion module. An output terminal of the multi-granularity featurefusion module is connected to an input terminal of the disease type classifier module.

The data preprocessing module is configured to manually annotate medical text data according to a target category to be predicted, and load the medical text data into a domain ontology, and is also configured to segment the medical text data with Chinese character strings according to punctuation marks, numbers, and space characters, and remove off-stream words.

The featureextraction module is configured to extract an entity representation, a word representation, a semantic relation representation, and an attribute-value pair representation in the medical text data.

The multi-granularity featurefusion module is configured to fuse entity embedding feature, word embeddings feature, semantic relation representation, and attribute-value pair representation as inputs of softmax layer for disease prediction. The disease type classifier module is configured to generate a classification result of a disease type.

Preferably, the featureextraction module further includes four submodules, namely: an entity embedding representation feature module, a word feature embedding representation module, a semantic relation representation module, and an attribute-value pair extraction module.

The entity embedding representation module is connected to the word embedding representation module. The word feature extraction module is connected to the attribute-value pair extraction module. The attribute-value pair extraction module is connected to the semantic relation representation extraction module.

The entity embedding representation module is configured to map a processed medical text to the medical ontology, extract a concept's own feature and a concept type feature, and combine the concept's own feature and the concept type feature to extract a concept feature.

The word feature extraction module is configured to perform BiGRU learning of a word sequence feature in context for the concept, where the concept cannot be found to match the word feature extraction module from the medical ontology.

the semantic relation representation extraction module is configured to find an entity pair of a corresponding relation category in the domain ontology and an entity pair whose corresponding relation category cannot be found in the domain ontology.

The attribute-value pair extraction module is configured to extract a relation between disease-time and a detection-examination result.

Compared with the prior art, the present invention has the following beneficial effects:

Traditional methods mostly consider that words, characters or entity vectors cannot fully understand information expressed in a medical text, and much disease-related information is hidden in a semantic relation between entities or the words. The present invention can not only learn an entity reorientation or a word representation, but also mine a deeper semantic relation representation and an attribute-value pair. Then, features of different granularities are fused to improve the semantic reasoning ability of a model.

### BRIEF DESCRIPTION OF THE FIGURES

In order to explain embodiments of the present invention or the technical solutions in the prior art more clearly, the following briefly introduces the drawings that need to be used in the embodiments. Obviously, the drawings in the following description are only some of embodiments of the present invention. The person skilled in the art can obtain other drawings based on these drawings without creative work.

FIG. 1 is a schematic diagram of a flowchart of a method of the present invention; and

FIG. 2 is a schematic diagram of modules of a system of the present invention.

### DETAILED DESCRIPTION OF THE PREFERRED EMBODIMENTS

The following clearly and completely describes the technical solutions in embodiments of the present invention in conjunction with the drawings in the embodiments of the present invention. Obviously, the described embodiments are only a part of the embodiments of the present invention, rather than all embodiments. Based on the embodiments of the present invention, all other embodiments obtained by the person skilled in the art without inventive work shall fall within the protection scope of the present invention.

In order to make the forgoing objectives, features and advantages of the present invention more obvious and easy to understand, the present invention is further described in detail with reference to the drawings and specific embodiments.

### Embodiment 1

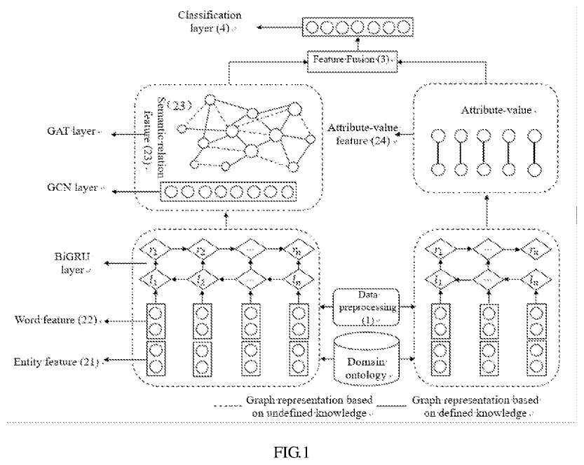

Referring to FIG. 1, the present invention proposed a medical prediction method based on a semantic graph network, specifically including the following steps: S1. Manually labeling medical text data according to a target category to be predicted; then loading the medical text data into the domain ontology; dividing a text to be processed into Chinese character strings according to punctuation marks, numbers and space characters; and removing off-stream words.

S2. Performing entity embedding representation (21), word embedding representation (22), semantic relation representation extraction (23), and attribute-value pair extraction (24) on the preprocessed medical text data.

The entity embedding representation (21): an entity representation included an entity representation and an entity type representation. First, the preprocessed text was mapped to the domain ontology, and the text data was divided into a semantic set {Y₁, . . . Yₙ}∈D (D was a text data) via a maximum matching method, where D included an entity set {C₁, . . . Cₙ}∈Y and had a corresponding entity type {C_{1type}, . . . C_{Ntype}}, and an entity set could be found in the domain ontology. An entity representation was extracted by combining the entity representation and the entity type representation, and denoted as eᵢ=cᵢ⊕c_{itype}e={e₁ . . . eₙ}eᵢ∈e, where cᵢ was the concept's own feature and belonged to a concept set {C₁ . . . C_{N}}. c_{itype} was the concept cᵢ's type feature and belonged to {C_{1type} . . . C_{Ntype}}, and ⊕ was a vector splicing operation. In this method, both the entity and a word belonged to a word-level feature. The word2vec model was used to convert the entity, the entity type and a word in a context into a d-dimensional vector form. Graph representation methods of the entity and the word were introduced in a graph representation based on undefined knowledge method in (23).

The word embedding representation (22): Bi-GRU was used to capture a dependency relation between word sequences and extract a word representation. If there was a word sequence wₗ∈[w₁, . . . , wₙ] and the corresponding hidden unit hᵢ∈[h, . . . , hₙ], context information of the word sequence and a corresponding hidden unit might be obtained by formula (1) and formula (2):

\vec{hᵢ}=\vec{GRU}(wᵢ,θ),i∈[1,n] (1)

[symbol]

= [symbol] (wᵢ,θ),i∈└n,1┘ (2)

θ represented parameters in a GRU model. Forward sequence information \vec{hᵢ} and reverse sequence information [symbol] were combined to extract a context feature hᵢ=[\vec{h}, [symbol] ] of the word wᵢ, where hᵢ represented a hidden state. Finally, the [symbol] sequence information between the words was put into a graph attention network to identify a semantic relation and extract an attribute-value pair.

The semantic relation representation extraction (23): in this step, the present invention used a graph convolution network and the graph attention network to construct a semantic relation graph and define two types of subgraphs: (1) graph representation based on defined knowledge: the subgraph used a relation between entities marked in the domain ontology, and used the graph convolution network and the graph attention network to extract a graph representation of an entity relation in an electronic medical record text. (2) Graph representation based on undefined knowledge: for an entity or a word (where the entity or the word could not be found in the domain ontology), according to a dependency relation between words in context extracted by the Bi-GRU, the graph convolution network and the graph attention network were directly used to extract a relation between the words or the entities.

(1) The graph representation based on defined knowledge: first, based on a medical ontology, entities contained in an electronic medical record and the relation between the entities were identified as a node and an edge of a graph, where the node and the edge were recorded as V^{K} and E^{K}, respectively. {h₁, h₂, . . . , h_{|n|}} was used to represent a feature of the node {v₁, v₂, . . . , v_{|n|}}, hᵢ∈ [symbol] , eᵢⱼ^{r}=(vᵢ,vⱼ), where, i≠j indicated that there was a corresponding relation r of the node vᵢ and vⱼ in an ontology. Then a knowledge graph representation model G^{K}={V^{K},E^{K}} was built based on |V^{K}| and |E^{K}|. Due to individual differences in patients, a fine-granularity relation between the entities could provide more detailed disease-related information and was more important for disease prediction. However, the same entity pair might correspond to a variety of different relations in the domain ontology. For example, there might be a relation TrID (a treatment method improved a certain disease) between a disease entity “chronic constipation” and a treatment entity “Dumic”, where a TrWD treatment method worsened a certain disease, and was applied to a certain disease, and a treatment effect was not stated. Therefore, the present invention used syntactic analysis to extract a trigger word and an adjective of the trigger word and combine the trigger word and the adjective of the trigger word, and then used a cosine distance to calculate semantic similarity with a relation category, thereby determining which fine-granularity relation the entity pair belonged to. If there was not the adjective of the trigger word in a sentence, similarity between the trigger word and an entity category was directly calculated, as shown in formulas (3) and (4):

p₂=sim[(cᵢ⊖fᵢ)rᵢ] (3)

p₂=sim[cⱼ,rⱼ] (4)

Where, cᵢ and cⱼ represented the trigger words, fᵢ represented the adjective of cᵢ, rᵢ and rⱼ represented relation categories, and sim[a,b] represented the calculation of similarity between a and b. The present invention tested a similarity threshold value in the range of 0.85-0.92 in an experiment, and results showed that there was a best effect at 0.89.

Next, an adjacency matrix A^{K} was defined. For each graph, the present invention defined a binary matrix P∈ [symbol] ^{nd×nb} to represent the relation between the entities in the sentence. If the entity pairs vᵢ and vⱼ in the sentence had a corresponding entity relation in the domain ontology, then Pᵢⱼ=1, otherwise, Pᵢⱼ was equal to 0. The present invention only considered a first-order neighbor, and a knowledge-based adjacency matrix was represented by formula (5):

$${A}^{K} = [ \begin{matrix}0 & P \\ {P}^{T} & 0\end{matrix} ] \qquad ( 5 )$$

After obtaining the adjacency matrix, the present invention first used learning node representation of the graph convolution network, as shown in formula 6-2:

H^{K(t)}=ReLU(A^{K}H^{K(t-2)}W^{K(t-1)}+B^{K}) (6)

Where,

$${\tilde{A}}^{K} = {D}^{K - \frac{1}{2}} {A}^{K} {D}^{K - \frac{1}{2}} ,$$

D^{K} was a degree matrix of A^{K}, and the degree matrix is a diagonal matrix Dₜᵢ^{K}=Σⱼ₌₁ⁿ Aᵢⱼ^{K}. W^{K} and B^{K} represented a weight and bias parameters, W^{K}∈ [symbol] ^{(nd+nb)×l}, B^{K}∈ [symbol] ^{(nd+nb)×l}. ReLU represented a nonlinear activation function. H^{K(t-1)} represented a feature of a previous layer of H^{K}.

After a graph convolution layer, the present invention combined the entity relation in the domain ontology and used a graph attention layer to extract knowledge-based node representation. For a given node, the graph attention network first learned the importance of a neighboring node with the same relation and fused the neighboring node according to a weight score. If there were node features h−{h₁, h₂, . . . , h_{|n|}} and hᵢ∈ [symbol] ^{F}, a new node representation set was generated as an output h={h₁′, h₂′, . . . , h_{|n|}′}, hᵢ′∈ [symbol] ^{F′} via the graph attention layer. F′ represented the dimension of an output feature. In order to transform an input into a higher-level output feature, the graph attention layer used a weight matrix to parameterize shared linear transformation at each node, W∈ [symbol] ^{F′×F} and used a shared attention mechanism to calculate an attention coefficient, as shown in formula (7):

eᵢⱼ^{Φr}=a(W_{b}hᵢ,W_{b}(hⱼ|Eᵣ)) (7)

Where, eᵢⱼ^{Φr} represented that graphs Φ consisting of entity pairs vᵢ and vⱼ in the sentence had a relation in the domain ontology r. Eᵣ represented a relation vector of r. W_{b} represented a weight. a∈ [symbol] ^{2F′}. Next, the present invention used formula (8) to regularize weight scores of the adjacent nodes:

$${α}_{ij}^{Φ r} = \frac{exp ( {e}_{ij}^{Φ r} )}{{\sum }_{K ∈ {N}_{i}^{Φ r} exp ( {e}_{ij}^{Φ r} )}} \qquad ( 8 )$$

Where, Nᵢ^{Φr} represented the neighbor node of a node vᵢ and had a relation r. Finally, the feature of a subsequent node vᵢ was obtained by combining a knowledge graph with formula (9). X^{Φ}={x₁^{Φ}, . . . , xₙ^{Φ}}, xᵢ^{Φ}⊂x^{Φ} was used to represent a knowledge graph contained in an electronic medical record. {x₁^{Φ}, . . . , xₙ^{Φ}} Was combined to obtain the knowledge graph G^{K} of the electronic medical record, as shown in formula (10):

$${\text{?}}_{i}^{Φ} = ReLU ( {\sum }_{j ∈ {N}_{\text{?}}^{Φ r}} {\text{?}}_{ij}^{Φ r} {h}_{j} ) \qquad ( 9 )$$

$${G}^{K} = {\sum }_{i = 1}^{n} {x}^{Φ} \qquad ( 10 )$$

$$\text{?} \text{indicates text missing or illegible when filed}$$

(2) The graph representation based on undefined knowledge

For an entity or a word whose corresponding relation category could not be found from the ontology, a dependency relation between the word sequences was extracted according to the Bi-GRU, and the present invention used a graph convolution model to extract the graph representation based on undefined knowledge G^{C}={V^{C},E^{C}}. The adjacency matrix A^{C} was represented by formula (11). If the word or an entity node vₚ is related to v_{q}, where p=q or p≠q (when p=q, learning the feature of the concept or the word itself), then Uᵢⱼ=1, otherwise, Uᵢⱼ is equal to 0.

$$\begin{matrix}{\text{?}}^{C} = [ \begin{matrix}0 & M \\ {M}^{T} & 0\end{matrix} ] & ( 11 )\end{matrix}$$

$$\text{?} \text{indicates text missing or illegible when filed}$$

The learning node representation of the graph convolution network is shown in formula (12):

H^{C(t)}−ReLU(Ã^{C}H^{C(t-1)}W^{C(t-1)}+B^{C}) (12)

Where,

$${\text{?}}^{C} = {D}^{C - \frac{1}{2}} {\text{?}}^{C} {D}^{C - \frac{1}{2}} ,$$

$$\text{?} \text{indicates text missing or illegible when filed}$$

D^{C} was a degree matrix of A^{C}, and the degree matrix was a diagonal matrix Dᵢᵢ^{C}=Σⱼ₌₁ⁿ Aᵢⱼ^{C}. W^{C} and B^{C} represented the weight and the bias parameters. Then the graph attention network was used to update representation of the node vₚ, as shown in formula (13):

e_{pq}^{Φ}=a(Wⱼhₚ,Wⱼh_{q}) (13)

Next, formula (14) was used to regularize the weight scores of the adjacent nodes, and finally formula (15) was used to calculate the graph representation of the entity or the word vₚ and v_{q}.

$${α}_{pq}^{Φ} = \frac{exp ( LeakyRelu ( {α}^{T} \vert {We}_{p} \vert \vert {We}_{q} \vert ) )}{{\sum }_{g ∈ {N}_{j}^{Φ}} exp ( LeakyRelu ( {α}^{T} [ {We}_{p} \vert \vert {We}_{q} ) )} \qquad ( 14 )$$

$${z}_{j}^{Φ} = ReLU ( {\sum }_{q ∈ {N}_{j}^{Φ}} {α}_{pq}^{Φ} {h}_{q} ) \qquad ( 15 )$$

Where, ∥ represented the vector splicing operation. LeakyRelu represented a non-linear activation function. Nⱼ represented the neighbor node of vₚ. z^{Φ}={z₁^{Φ}, . . . , zₘ^{Φ}}, zⱼ^{Φ}∈z^{Φ} represented a text graph contained in the electronic medical record. A set graph {z₁^{Φ}, . . . , zₘ^{Φ}} obtained text graph representation G^{C}, as shown in formula (16).

G^{C}=Σⱼ₌₁^{m}z^{Φ} (16)

The attribute-value pair extraction (24): an attribute-value could be divided into two types: disease-time and a test-test result. where, the type of a disease-time value included only a numeric type, and the type of a test-test result value included the numeric type and a categorical type. Each attribute-value included two elements, an attribute and its corresponding value. Unlike an entity relation where a tail entity was usually relatively stable and would not change from a patient to a patient, in the attribute-value, the value would vary from a patient to a patient; for example, the blood pressure value of each patient was different. For the numeric type, each value could be expressed in different units, such as “10 years” and “122/70 mmHg”. For this type, the present invention first extracted a real value of EMR and its corresponding unit symbol, including a ratio symbol, such as “47.6%”, and a character symbol, such as “5 years”. If there were a real value Dᵢ and its corresponding unit symbol Uᵢ, the updated value could be represented by vᵢ=DᵢΦuᵢ (uᵢ was unit symbols). A categorical type value was considered to be word-level representation, and did not have the unit symbol. Due to the different expressions of different doctors, negative words contained in the electronic medical record usually changed the polarity of the categorical value; for example, the expressions of “not abnormal” and “normal” in “a patient's cardiac ultrasound was not abnormal” and “the patient's cardiac ultrasound was normal” had the same meaning. Therefore, it was necessary to combine the negative words to extract a categorical value feature. If there was no negative word prefix before the type value, word vector representation of the type value was directly extracted. If the type value was prefixed by a negative word, the present invention first combined the negative word with the type value, and then calculated similarity between the type value and other type values via the cosine distance (here a similarity distance was also set to 0.9).

According to the guidance of a medical expert, a quantitative threshold value was set for the value of each examination result during training for disease inference. The value of the examination result was divided into 4 levels: a low level, a normal level, a high level, and a very high level. If there was an examination entity vₙ, its corresponding examination result vₘ and grade index lᵢ, i=4 as well as the attribute-value of the test-test result could be expressed as a graph gₙ^{Φ}−[vₙ;(vₘ+lᵢ)], where [x₁;x₂] represented that vector splicing of x₁ and x₂ was performed. For the disease-time, if there was a disease entity vₒ and its corresponding time vₛ, an attribute-value of the disease-time could be expressed as gₒ^{Φ}=[vₒ;vₛ]. In addition, the expression of an attribute-value relation in the test-test result was the same as that of the disease-time. gₖ^{Φ} was used to represent one of the graphs in the attribute-value. gₖ^{Φ}∈{g₁^{Φ}, . . . , gₗ^{Φ}} obtained the graph of the attribute-value in a document, as shown in formula (17).

G^{V}=Σₖ₌₁^{l}g^{Φ} (17)

In the process of extracting an attribute-value pair, the present invention first identified a numerical value and a categorical value contained in a sentence, then learned context information of the value via the Bi-GRU, and extracted the entity closest to the value as its corresponding attribute feature.

S3. obtaining a final document feature representation dᵢ, i∈[1 . . . n] by combining the graph representation based on defined knowledge, the graph representation based on undefined knowledge and an attribute-value-based graph representation, as shown in formula (18).

dᵢ=[G^{K}⊕G^{C}⊕G^{V}] (18)

Where, G^{K} was knowledge graph representation, G^{C} was text graph representation, G^{V} was attribute-value graph representation, and ⊕ was the vector splicing operation.
S4. using the document feature representation d as an input of softmax layer to predict the level of COPD on the document, and calculating a loss function based on a cross entropy between a real label and a predicted label, as shown in formula (19) and formula (20).

$${\hat{y}}_{i} = p ( y \vert {d}_{i} ) = \frac{1}{1 - exp - ( {W}_{\text{?}} {d}_{\text{?}} + {b}_{e} )} ∈ \vert 0 , 1 {\vert}^{\text{?}} \qquad ( 19 )$$

$$ℒ ( θ ) = - \frac{1}{M} {\sum }_{i = 1}^{M} \text{?} ( {y}_{i} , {\hat{y}}_{i} ) \qquad ( 20 )$$

$$\text{?} \text{indicates text missing or illegible when filed}$$

Where, W_{c} and b_{c} represented a weight matrix and a bias term in a classification layer. θ represented the parameters in the model, including W^{k}, W^{c}, Wₑ. c represented the number of categorical labels, c>1. [symbol] represented the cross entropy between the real label yᵢ and the predicted label ŷᵢ.

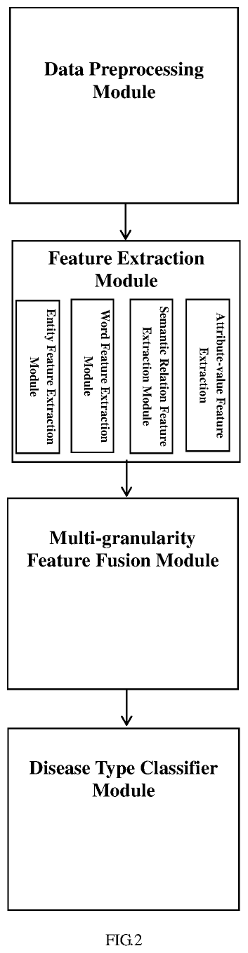

Referring to FIG. 2, the present invention proposed a medical prediction system based on a semantic graph network, including: a data preprocessing module, a feature extraction module, a multi-granularity feature fusion module, and a disease type classifier module.

An output terminal of the data preprocessing module is connected to an input terminal of the feature extraction module. An output terminal of the feature extraction module is connected to an input terminal of the multi-granularity feature fusion module. An output terminal of the multi-granularity feature fusion module is connected to an input terminal of the disease type classifier module.

The data preprocessing module was configured to manually label medical text data according to a target category to be predicted, then load the medical text data into a domain ontology; divide a text to be processed into Chinese character strings according to punctuation marks, numbers and space characters, and remove off-stream words.

The feature extraction module was divided into four submodules, namely: an entity embedding representation module, a word embedding representation module, a semantic relation representation extraction module, and an attribute-value pair extraction module.

(1) The entity embedding representation module was configured to map a processed medical text to a medical ontology, extract a concept's own feature and a concept type feature, and combine the concept's own feature and the concept type feature to extract a concept feature.
(2) The word embedding representation module was configured to use BiGRU to learn a sequence feature of a word in context if a concept matching the medical ontology could not be found from the medical ontology.
(3) The semantic relation representation extraction module: semantic relation included three types: an entity-entity relation, an entity-word relation, and a word-word relation. The entity-entity relation could be divided into two types, graph representation based on defined knowledge (referring to an entity pair, where the entity pair could find a corresponding relation category in the domain ontology) and the graph representation based on undefined knowledge (referring to an entity pair, where the entity pair could not find the corresponding relation category in the domain ontology). The word was not a medical term but included important semantic information (such as basic patient information). In a graph representation based on undefined knowledge, this method allowed to extract the relation between the entity or the word and the graph representation based on undefined knowledge, and graph representation of the entity or the word.
(4) The attribute-value pair extraction module: an attribute-value pair included two categories: disease-time and a test-test result. An attribute referred to an entity representation in Step (21). A value could be divided into two types: a numeric type value and a categorical type value. A value in the disease-time only included the numeric type value, and a value in the detection-examination result included the numeric type value and the category type value. Attribute-value graph representation was constructed according to each attribute and its corresponding value.

The multi-granularity feature fusion module was configured to fuse an extracted entity representation, an extracted word representation, an extracted semantic relation representation, and an extracted attribute-value pair representation as inputs of softmax layer for disease prediction. In order to prevent overfitting, a convolution layer of a graph convolution network used dropout operation and used zero padding to maintain the validity of a sentence.

The disease type classifier module was configured to put a result of model training into softmax classification layer, and use softmax classifier to generate a classification result of the final disease type.

The forgoing embodiments only describe the preferred mode of the present invention, and do not limit the scope of the present invention. Without departing from the design spirit of the present invention, the person skilled in the art can make variations and improvements to the technical solutions of the present invention, which should fall within the protection scope determined by the claims of the present invention.

What is claimed is:

1. A medical prediction method based on a semantic graph network, specifically comprising the following steps:
S1. preprocessing medical text data;
S2. Feature extraction on the preprocessed medical text data;
S3. fusing a multi-granularity feature on the extracted feature to obtain a final document feature representation; and
S4. predicting a chronic disease on the final document feature representation.

2. The medical prediction method based on the semantic graph network according to claim 1, wherein Step S1 is specifically as follows:
S11. manually annotating the medical text data according to a target category that needs to be predicted, and loading the medical text data into a domain ontology;
S12. cutting the medical text data into Chinese character strings according to punctuation marks, numbers and space characters, and removing off-stream words.

3. The medical prediction method based on the semantic graph network according to claim 1, wherein the feature extraction in Step S2 includes: entity embedding representation, word embedding representation, semantic relation representation extraction, and attribute-value pair extraction.

4. The medical prediction method based on the semantic graph network according to claim 3, wherein the entity extraction is specifically as follows:
first, mapping the preprocessed medical text data to the domain ontology; dividing the medical text data into semantic sets via a maximum matching method; then finding an entity set matching the semantic set and an entity type set corresponding to the entity set from the semantic set to obtain an entity representation and an entity type representation; and finally, combining the entity representation and the entity type representation to extract an entity representation.

5. The medical prediction method based on the semantic graph network according to claim 3, wherein the word embedding representation and the attribute-value pair extraction are specifically as follows:
using a Bi-GRU to find a dependency relation between word sequences in the medical text data, and putting sequence information between words into a graph attention network to identify semantic relation and extract an attribute-value pair.

6. The medical prediction method based on a semantic graph network according to claim 3, wherein the semantic relation representation extraction is specifically as follows:
using a graph convolution network and the graph attention network to construct a semantic relation graph and defining two types of subgraphs of graph representation based on defined knowledge and graph representation based on undefined knowledge, wherein the graph representation based on defined knowledge uses a relation between entities marked in the domain ontology and uses the graph convolution network and the graph attention network to extract an entity relation in an electronic medical record text, for the entity or the word whose corresponding relation cannot be found from the domain ontology, the graph representation based on undefined knowledge directly uses the graph convolution network and the graph attention network to extract a relation between the words or the entities based on a dependency relation between words in context extracted by the Bi-GRU.

7. The medical prediction method based on the semantic graph network according to claim 1, wherein Step S3 is specifically as follows:
feature-fusing an extracted entity representation, an extracted word representation, an extracted semantic relation representation, and an attribute-value pair representation to obtain the final document feature representation.

8. The medical prediction method based on the semantic graph network according to claim 1, wherein Step S4 is specifically as follows:
inputting the document feature representation into softmax layer for medical prediction, and calculating a loss function based on a cross entropy between a real label and a predicted label to obtain a classification result of a disease type and a prediction result of a disease level.

9. A medical prediction system based on a semantic graph network, comprising a data preprocessing module, a feature extraction module, a multi-granularity feature fusion module, and a disease type classifier module;
an output terminal of the data preprocessing module is connected to an input terminal of the feature extraction module; an output terminal of the feature extraction module is connected to an input terminal of the multi-granularity feature fusion module; an output terminal of the multi-granularity feature fusion module is connected to an input terminal of the disease type classifier module;
the data preprocessing module is configured to manually annotate medical text data according to a target category to be predicted, and load the medical text data into a domain ontology, and is also configured to segment the medical text data with Chinese character strings according to punctuation marks, numbers, and space characters, and remove off-stream words;
the feature extraction module is configured to extract an entity representation, a word representation, a semantic relation representation, and a attribute-value pair in the medical text data;
the multi-granularity feature fusion module is configured to fuse an extracted entity representation, an extracted word representation, an extracted semantic relation representation, and an attribute-value pair representation as inputs of softmax layer for disease prediction;
the disease type classifier module is configured to generate a classification result of a disease type.

10. The medical prediction system based on the semantic graph network according to claim 9, wherein the feature extraction module further includes four sub-modules, namely: an entity embedding representation module, a word embedding representation module, and a semantic relation representation extraction module and an attribute-value pair extraction module;
the entity embedding representation module is connected to the word feature extraction module, the word embedding representation module is connected to the attribute-value pair extraction module, the attribute-value pair extraction module is connected to the semantic relation representation extraction module;
the entity embedding representation module is configured to map a processed medical text to the medical ontology, extract a concept's own feature and a concept type feature, and combine the concept's own feature and the concept type feature to extract a concept feature;
the word embedding representation module is configured to perform BiGRU learning of a word sequence feature in context for the concept, wherein the concept cannot be found to match the word embedding representation module from the medical ontology;
the semantic relation representation extraction module is configured to find an entity pair of a corresponding relation category in the domain ontology and an entity pair whose corresponding relation category cannot be found in the domain ontology;
the attribute-value pair extraction module is configured to extract a relation between a disease-time and a detection-examination result.
